(19) United States
(12) Patent Application Publication
(10) Pub. No.: US 2025/0360120 A1
(43) Pub. Date: Nov. 27, 2025

(54) METHODS OF TREATMENT OF DRAVET SYNDROME

(71) Applicant: Noema Pharma AG, Basel (CH)

(72) Inventor: George Garibaldi, Basel (CH)

(21) Appl. No.: 19/287,511

(22) Filed: Jul. 31, 2025

Related U.S. Application Data
(63) Continuation of application No. PCT/US2024/014248, filed on Feb. 2, 2024.
(60) Provisional application No. 63/482,918, filed on Feb. 2, 2023.

(51) Int. Cl. A61K 31/4439; A61P 25/08; C07D 401/06

(52) U.S. Cl. CPC A61K 31/4439; A61P 25/08; C07D 401/06

(57) ABSTRACT

Provided herein are methods of treating Dravet syndrome in a subject in need thereof by administering to the subject compositions comprising an mGlu5 negative allosteric modulator (NAM).

### CROSS-REFERENCE TO RELATED APPLICATIONS

The application is a continuation of International Patent Application No. PCT/US2024/014248, filed Feb. 2, 2024, which claims the benefit of, and priority to, U.S. Patent Application No. 63/482,918, filed Feb. 2, 2023, the contents of each are hereby incorporated by reference in their entirety herein for all purposes.

### FIELD

The present disclosure relates to the field of medicine and the treatment of Dravet syndrome. More specifically, the present disclosure relates to use of compositions comprising 2-chloro-4-[1-(4-fluorophenyl)-2,5-dimethyl-1H-imidazol-4-ylethynyl]pyridine, or a pharmaceutically acceptable salt thereof, in the treatment or amelioration of Dravet syndrome.

### BACKGROUND

Dravet syndrome (DVS), a.k.a Severe Myoclonic Epilepsy of Infancy (SMEI), is a rare, catastrophic, lifelong form of epilepsy that begins in the first year of life with frequent or prolonged seizures. Children with DVS typically experience a lagged development of language and motor skills, hyperactivity and sleep difficulties, chronic infection, growth and balance issues, and difficulty relating to others. In 70-90% of patients, DVS is caused by the mutation of the SCN1A gene (sodium channel, voltage-gated, type I, alpha subunit), resulting in a premature stop codon and thus a non-functional protein. See, e.g., Selmer et al., Clinical Genetics., 2009, 76 (4): 398-403.

Current treatment options for DVS are limited. Seizures in DVS can be difficult to manage but may be reduced by antiseizure medications such as clobazam, stiripentol, topiramate, valproate, fenfluramine and cannabidiol. Yet, certain medications have been known to exacerbate seizures in patients due to their effects on the sodium ion channel. Also, some medications have significant side effects, thereby limiting their medical use.

Therefore, there is an unmet medical need to develop new methods for treating DVS without significant side effects.

### SUMMARY

The disclosure provides, in part, a method of treating Dravet syndrome (DVS), comprising administering to a subject in need thereof a composition comprising a therapeutically effective amount of a therapeutic agent, wherein the therapeutic agent is a compound of Formula I:

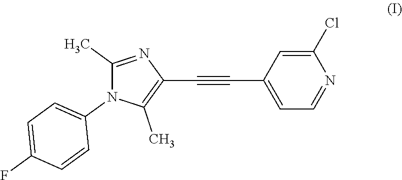 [SMILES: Cc1nc(C#Cc2ccnc(Cl)c2)c(C)n1-c1ccc(F)cc1]

or a pharmaceutically acceptable salt thereof.

Also provided herein, in part, is a method of treating Dravet syndrome, comprising administering to a subject in need thereof a composition comprising a therapeutically effective amount of an mGlu5 negative allosteric modulator (NAM), or a pharmaceutically acceptable salt thereof, wherein the mGlu5 NAM is a compound of Formula I:

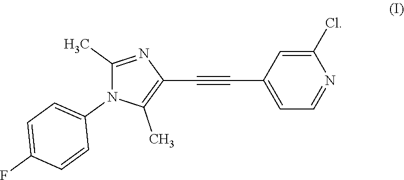 [SMILES: Cc1nc(C#Cc2ccnc(Cl)c2)c(C)n1-c1ccc(F)cc1]

### BRIEF DESCRIPTION OF THE DRAWINGS

FIG. 1 depicts an exemplary XRPD pattern of a crystalline anhydrate form (Form A) of a monosulfate of the compound of Formula I.

FIG. 2 depicts an exemplary XRPD pattern of a crystalline monohydrate form (Form B) of a monosulfate of the compound of Formula I.

FIG. 3 depicts an exemplary XRPD pattern of a crystalline hemihydrate form (Form C) of a hemisulfate of the compound of Formula I.

FIG. 4 depicts frequency and percent of animals exhibiting seizure freedom across Compound I 1 mg/kg, Compound 1 0.3 mg/kg, CTEP and Untreated groups.

FIG. 5 depicts a Kaplan-Meier plot that demonstrates how the percent of animals with seizure freedom changes over time for Compound I 1 mg/kg, Compound 1 0.3 mg/kg, CTEP and Untreated groups.

FIG. 6 depicts a plot of the individual total seizure burden data on the Compound I 1 mg/kg, Compound I 0.3 mg/kg, CTEP and Untreated groups.

FIG. 7 depicts a plot of adjusted data of FIG. 6 using a logarithmic scale (log₁₀) on the y axis.

FIG. 8 depicts total seizure burden (geometric means and 95% confidence intervals) for the Compound I 1 mg/kg, Compound 1 0.3 mg/kg, CTEP and Untreated groups.

FIG. 9 depicts a comparison against the Untreated group, expressed as ratios of geometric means and 95% confidence intervals.

FIG. 10 depicts individual seizure count per day data across Compound I 1 mg/kg, Compound I 0.3 mg/kg, CTEP and Untreated groups.

FIG. 11 depicts adjusted data of FIG. 10 using a logarithmic (log₁₀) scale on the y axis.

FIG. 12 and FIG. 13 depict results of an ANOVA performed on the log transformed adjusted data for average seizure per day. FIG. 13 contains the comparisons against the Untreated group, expressed as ratios of geometric means and 95% confidence intervals.

### DETAILED DESCRIPTION

As generally described herein, the present disclosure provides methods of treating Dravet syndrome (DVS) in a subject in need thereof with a compound of Formula I, or a pharmaceutically acceptable salt thereof. Also provided herein are methods of treating a symptom of Dravet syndrome, for example a seizure, in a subject in need thereof with a compound of Formula I, or a pharmaceutically acceptable salt thereof.

### Compound

The compound of Formula I, as depicted below, is an mGlu5 negative allosteric modulator (NAM), also known as 2-chloro-4-[1-(4-fluorophenyl)-2,5-dimethyl-1H-imidazol-4-ylethynyl]pyridine or Basimglurant:

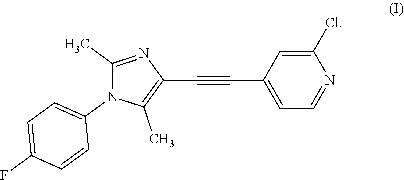 [SMILES: Cc1nc(C#Cc2ccnc(Cl)c2)c(C)n1-c1ccc(F)cc1]

A method of chemically synthesizing the compound of Formula I (including Example 1 provided herein) is described in U.S. Pat. No. 7,332,510, which is incorporated by reference in its entirety. As used herein, the compound of Formula I is referred to as “Compound I.”

It should be understood that the compound of Formula I as described herein includes crystalline solid forms of either the free base or pharmaceutically acceptable salts of the compound of Formula I as described herein.

In certain embodiments, the pharmaceutically acceptable salt of the compound of Formula I can be a salt of the compound of Formula I with physiologically compatible mineral acids, such as hydrochloric acid, sulfuric (or sulphuric) acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. An exemplary pharmaceutically acceptable salt of the compound of Formula I is a monosulfate salt or a hemisulfate salt.

In certain embodiments, the pharmaceutically acceptable salt of the compound of Formula I is a monosulfate salt or a hemisulfate salt, each being in hydrate or anhydrate form (e.g., anhydrate, hemihydrate, or monohydrate).

In certain embodiments, the pharmaceutically acceptable salt of the compound of Formula I is in a crystalline form or an amorphous form.

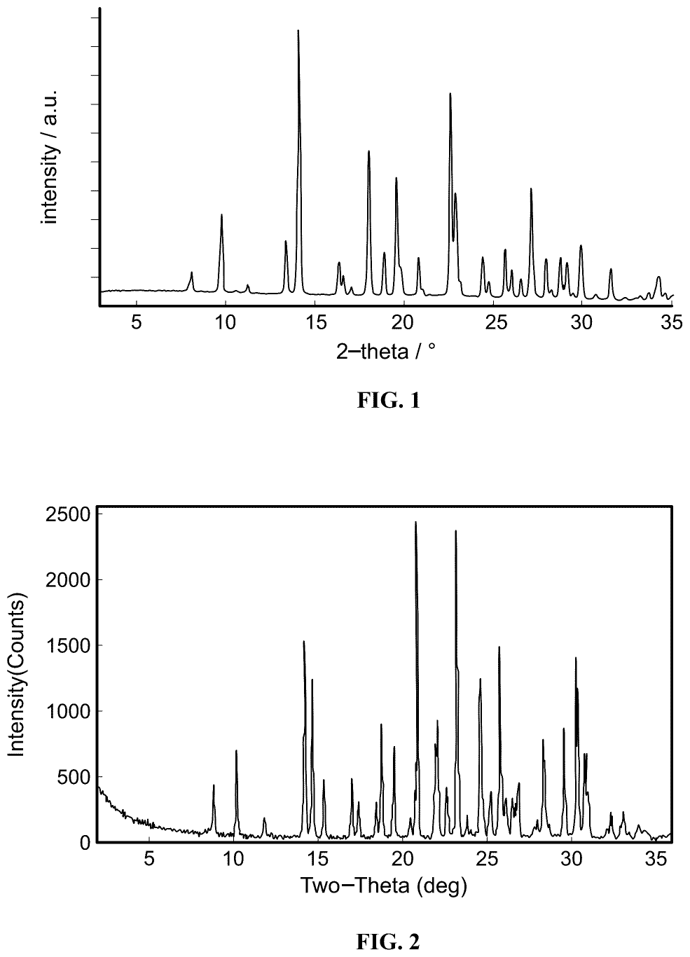

In some embodiments, the compound is in a crystalline anhydrate form (Form A) of a monosulfate salt of the compound of Formula I, wherein Form A has an X-ray powder diffraction (XRPD) pattern as substantially shown in FIG. 1. In some embodiments, Form A is characterized by at least three peaks selected from the following X-ray powder diffraction peaks obtained with a Cuka radiation at 2θ (2 Theta): 9.8, 13.4, 14.2, 18.1, 18.9, 19.6, 22.6, 22.9, 25.7, 27.1, and 29.9) (±0.2°. The crystalline Form A typically has a Tₘ of about 180-190° C. by DSC analysis. In some embodiments, Form A is characterized by an infrared spectrum having sharp bands at 3068, 2730, 2618, 2236, 2213,1628, 1587,1569, 1518, 1384,1374, 1295,1236, 1168, 1157, 1116, 1064, 1019, 902, 855, 786, and 674 cm⁻¹ (±3 cm⁻¹).

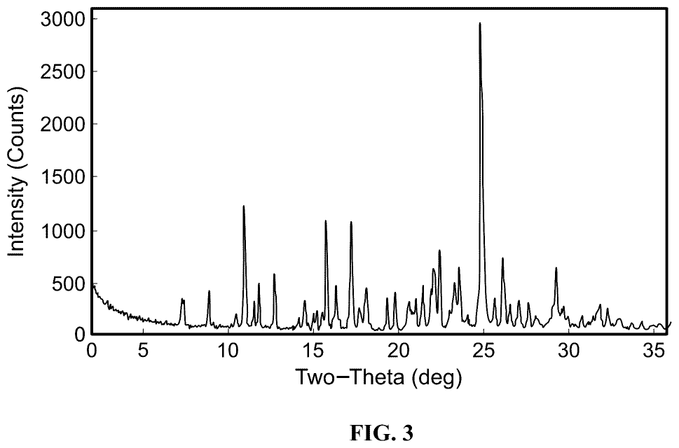

In some embodiments, the compound is in a crystalline monohydrate form (Form B) of a monosulfate salt of the compound of Formula I, wherein Form B has an XRPD pattern as substantially shown in FIG. 2. The crystalline Form B typically has a Tₘ of about 60-70° C. by DSC analysis.

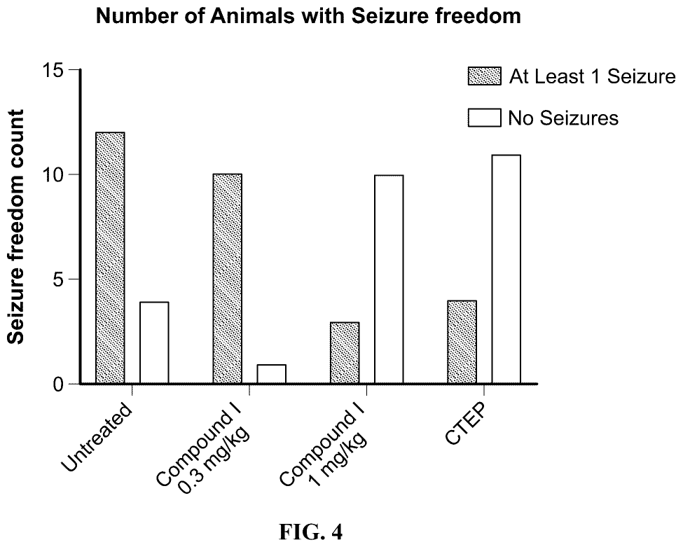

In some embodiments, the compound is in a crystalline hemihydrate form (Form C) of a hemisulfate salt of the compound of Formula I, wherein Form C has an XRPD pattern as substantially shown in FIG. 3. The crystalline Form C typically has a Tₘ of about 90-100° C. by DSC analysis.

### Methods of Use and Treatment

In one aspect, provided herein are methods of treating Dravet syndrome (DVS) in a subject in need thereof. Exemplary subjects include, but not limited to, a human and a patient, including infants, children, young adults, and adults. In an aspect, the disclosure provides a method of treating a patient diagnosed with Dravet syndrome which comprises administering to a patient a therapeutically effective dose of a compound of Formula I.

In another aspect, provided herein are methods of treating a symptom associated with Dravet syndrome (DVS) in a subject in need thereof. Exemplary subjects include, but not limited to, a human, a participant, and a patient, including infants, children, young adults, and adults. In an aspect, the disclosure provides a method of treating a patient exhibiting a symptom associated with Dravet syndrome which comprises administering to a patient a therapeutically effective dose of a compound of Formula I. In embodiments, the symptom is a seizure. In embodiments, the Dravet syndrome is associated with multiple types of seizures, and the subject (e.g., human, patient or participant) experiences multiple types of seizures. Thus, in various embodiments, provided herein are methods of treating a seizure associated with Dravet syndrome (DVS) in a subject in need thereof.

Dravet syndrome (DVS) is a catastrophic type of epilepsy with prolonged seizures that are often triggered by hot temperatures or fever. DVS has been characterized by prolonged febrile and non-febrile seizures within the first year of a child's life. Common issues associated with DVS include: prolonged seizures; frequent seizures; behavioral and developmental delays; movement and balance issues; orthopedic conditions; delayed language and speech issues; growth and nutrition issues; sleeping difficulties; chronic infections; sensory integration disorders; dysautonomia, or disruptions of the autonomic nervous system which can lead to difficulty regulating body temperature, heart rate, blood pressure, and other issues.

In various embodiments, provided herein are methods for treating Dravet syndrome (DVS), comprising administering to a subject in need thereof a composition comprising a therapeutically effective amount of a therapeutic agent, wherein the therapeutic agent is a compound of Formula I:

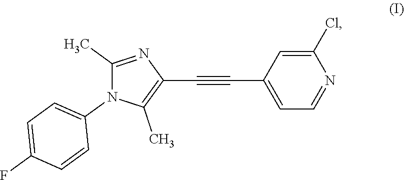 [SMILES: Cc1nc(C#Cc2ccnc(Cl)c2)c(C)n1-c1ccc(F)cc1]

or a pharmaceutically acceptable salt thereof.

In various embodiments, provided herein are methods for treating a symptom (e.g., a seizure) associated with Dravet syndrome (DVS), comprising administering to a subject in need thereof a composition comprising a therapeutically effective amount of a therapeutic agent, wherein the therapeutic agent is a compound of Formula I:

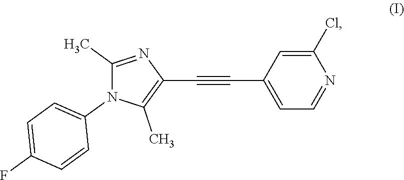 [SMILES: Cc1nc(C#Cc2ccnc(Cl)c2)c(C)n1-c1ccc(F)cc1]

or a pharmaceutically acceptable salt thereof.

In various embodiments, provided herein is a use of a compound or a pharmaceutically acceptable salt thereof, in the manufacture of a medicament for treating a medical condition associated with Dravet syndrome (DVS) in a subject, wherein the compound is of Formula I:

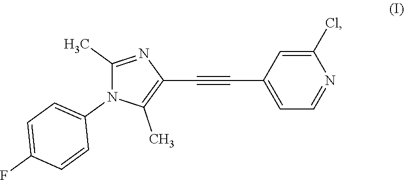 [SMILES: Cc1nc(C#Cc2ccnc(Cl)c2)c(C)n1-c1ccc(F)cc1]

or a pharmaceutically acceptable salt thereof.

In various embodiments, administering a therapeutically effective amount of the compound of Formula I, or a pharmaceutically acceptable salt thereof, comprises administering to a subject from about 0.1 mg to about 5 mg (e.g., about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg) of the compound of Formula I, or a pharmaceutically acceptable salt thereof. In various embodiments, administering a therapeutically effective amount of the compound of Formula I, or a pharmaceutically acceptable salt thereof, comprises administering to a subject from about 0.5 mg to about 4 mg (e.g., about 0.5 mg, about 0.7 mg, about 1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1, about 3.2 mg, about 3.3 mg, about 3.4 mg, about 3.5 mg, about 3.6 mg, about 3.7 mg, about 3.8 mg, about 3.9 mg, about 4.0 mg).

In certain embodiments, administering a therapeutically effective amount of the compound of Formula I, or a pharmaceutically acceptable salt thereof, comprises administering from about 0.5 mg to about 3.5 mg of the compound of Formula I, or a pharmaceutically acceptable salt thereof, to a subject in need thereof. In certain embodiments, administering a therapeutically effective amount of the compound of Formula I, or a pharmaceutically acceptable salt thereof, comprises administering from about 0.5 mg, about 0.8 mg, about 1.0 mg, about 1.3 mg, about 1.5 mg, about 1.8 mg, about 2 mg, about 2.3 mg, about 2.5 mg, about 2.8 mg, about 3 mg, about 3.3 mg, about 3.5 mg, about 3.8 mg, or about 4.0 mg of the compound of Formula I, or a pharmaceutically acceptable salt thereof, to a subject in need thereof.

In some embodiments, the compound of Formula I is dosed at about 0.3 mg/kg. In some embodiments, the compound of Formula I is dosed at about 1.0 mg/kg.

In some embodiments, the subject has a weight of at least 40 kg. In some embodiments, the subject has a weight of less than 40 kg. For instance, Compound I dosing may follow two weight categories, (<40 kg and ≥40 kg). The initial dose of Compound I may be 0.5 mg or 1.0 mg once daily for patients weighing <40 kg, and 1.5 mg once daily for patients weighing ≥40 kg. The maximum doses of Compound I are 3.0 mg once daily for patients <40 kg, and 3.5 mg once daily for patients ≥40 kg. Thereafter, the dose of Compound I may be escalated in a blinded fashion in 0.5 mg increments at weekly intervals according to individual tolerability.

In certain embodiments, provided herein is a method of administering the free base form of the compound of Formula I for the treatment of DVS in a subject in need thereof.

In certain embodiments, provided herein is a method of administering a pharmaceutically acceptable salt of the compound of Formula I for the treatment of DVS in a subject in need thereof. In certain embodiments, as described above, treating comprises administering the compound of Formula I in a crystalline form (e.g., Form A, Form B, or Form C) in a sulfate salt form (e.g., a monosulfate salt or a hemisulfate salt). In some embodiments, the pharmaceutically acceptable salt of the compound of Formula I can be a salt of the compound of Formula I with physiologically compatible mineral acids, such as hydrochloric acid, sulfuric (or sulphuric) acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid.

In certain embodiments, treating comprises administering the compound of Formula I, or a pharmaceutically acceptable salt thereof, once daily.

In certain embodiments, treating comprises administering the compound of Formula I, or a pharmaceutically acceptable salt thereof, by oral administration.

In certain embodiments, treating comprises administering the compound of Formula I, or a pharmaceutically acceptable salt thereof, as a unit dose.

In some embodiments, provided herein are methods of treating DVS, comprising administering to a subject in need thereof a composition comprising a therapeutically effective amount of an mGlu5 negative allosteric modulator (NAM), or a pharmaceutically acceptable salt thereof, wherein the mGlu5 NAM is a compound of Formula I:

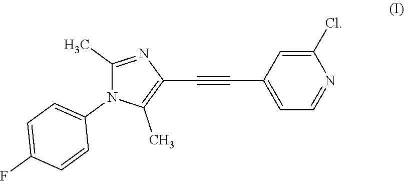 [SMILES: Cc1nc(C#Cc2ccnc(Cl)c2)c(C)n1-c1ccc(F)cc1]

In certain embodiments, the method comprises administering the compound of Formula I as a monotherapy.

In some embodiments, the method further comprises administering a second therapy for Dravet Syndrome. In some embodiments, the method further comprises administering a second therapy for seizure. In some cases, the subject is undergoing a second therapy for Dravet Syndrome or seizure.

In embodiments, methods described herein may reduce the frequency of seizures, reduce the severity of seizures, change the type of seizures (e.g., from a more severe type to a less severe type), or a combination thereof in a subject after treatment compared to the absence of treatment (e.g., before treatment), or compared to treatment with an alternative conventional treatment. In an aspect, the administration of the compound of Formula I, or a pharmaceutically acceptable salt thereof, or its crystalline form, described herein reduces convulsive seizure frequency by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, 100 days or more. the administration of the compound of Formula I, or a pharmaceutically acceptable salt thereof, or its crystalline form, described herein reduces non-convulsive seizure frequency by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, 100 days or more. In some embodiments, the method further comprises repeating the administering until the subject is permanently seizure free.

As such, the compound of Formula I, or a pharmaceutically acceptable salt thereof, or its crystalline form, described herein can reduce a seizure type after administration to a subject in need thereof. The reduction may be of one, two, three or multiple specific types of seizures. In some embodiments, one seizure type is reduced. In some embodiments, two seizure types are reduced. In some embodiments, three seizure types are reduced. In some embodiments, multiple seizure types are reduced. In some embodiments of the method, the seizure type reduced is selected from the group consisting of non-convulsive seizures, generalized seizures, myoclonic seizures, absence/atypical absence seizures, and febrile seizures, or any combination thereof. In some embodiments, particularly in Dravet syndrome, multiple seizure types are typically present including convulsive seizures consisting of generalized clonic seizures (GCS), generalized tonic-clonic seizures (prior terminology was grand mal), or alternating unilateral clonic seizures; myoclonic seizures; atypical absences and obtundation (dulled or impaired awareness) status; focal seizures, with or without secondary generalization; or, more rarely, tonic seizures. In some embodiments, the seizure type reduced is selected from the group consisting of photosensitive seizures and self-induced seizures. In some embodiments, the seizure type reduced is selected from atonic, or focal seizures without clear observable motor signs.

Examples of seizures also include, but not limited to, focal onset seizure, generalized onset seizure, and unknown onset seizure.

In an embodiment, the focal onset seizure is selected from focal aware seizure and focal impaired awareness seizure. In an embodiment, the focal onset seizure is selected from focal motor onset seizure and focal nonmotor onset seizure. In embodiments, the focal motor onset seizure can be selected from the group consisting of focal motor automatisms seizure, focal motor atonic seizure, focal motor clonic seizure, focal motor epileptic spasm seizure, focal motor hyperkinetic seizure, focal motor myoclonic seizure, and focal motor tonic seizure, or combinations thereof. In embodiments, the focal nonmotor onset seizure can be selected from the group consisting of focal nonmotor autonomic seizure, focal nonmotor behavior arrest seizure, focal nonmotor cognitive seizure, focal nonmotor emotional seizure, and focal nonmotor sensory seizure, or combinations thereof. In some embodiments, the seizure is focal to bilateral tonic-clonic seizure.

In an embodiment, the generalized onset seizure is selected from generalized motor seizure and generalized nonmotor (absence) seizure. In embodiments, the generalized onset motor seizure is selected from the group consisting of generalized onset motor tonic-clonic seizure, generalized onset motor clonic seizure, generalized onset motor tonic seizure, generalized onset motor myoclonic seizure, generalized onset motor myoclonic-tonic-clonic seizure, generalized onset motor myoclonic-atonic seizure, generalized onset motor atonic seizure, and generalized onset motor epileptic spasm seizure. In embodiments, the generalized onset nonmotor (absence) typical seizure, generalized onset nonmotor (absence) atypical seizure, generalized onset nonmotor (absence) myoclonic seizure, and generalized onset nonmotor (absence) eyelid myoclonia seizure.

In an embodiment, the unknown onset seizure is selected from unknown onset motor seizure and unknown onset nonmotor seizure. In an embodiment, the unknown onset motor seizure is selected from unknown onset motor tonic-clonic seizure and unknown onset motor epileptic spasm seizure. In an embodiment, the unknown onset nonmotor seizure is unknown onset nonmotor behavior arrest seizure.

In some embodiments, the seizure is an unclassified seizure.

In embodiments, the administration of the compound of Formula I, or a pharmaceutically acceptable salt thereof, or its crystalline form, described herein reduces status epilepticus frequency by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, 100 days or more.

In another aspect of the disclosure, the administration of the compound of Formula I, or a pharmaceutically acceptable salt thereof, or its crystalline form, described herein reduces the subject's hospitalization visits by about 25% or more, about 50% or more, about 75% or more, or completely eliminate hospitalization visits due to seizures.

In another aspect of the disclosure, the administration of the compound of Formula I, or a pharmaceutically acceptable salt thereof, or its crystalline form, described herein reduces the need by the subject for rescue medication by about 25% or more, about 50% or more, about 75% or more, or completely eliminate the need for rescue medication.

Seizures experienced in DVS are known to be extremely resistant to treatment. An effective treatment may be developed to reduce the number of seizures that can help make the condition more manageable. Further, children with DVS usually develop intellectual disabilities over time. They may have issues with expressing and understanding language. They may also have an increased risk of developing autism, attention deficit hyperactivity disorder (ADHD), and other behavioral problems. Children with Dravet Syndrome often require speech, occupational, and social and play therapy to support cognitive development. In certain embodiments, the therapeutic effect of the treatment of the present invention is determined by:
a) reduction of the overall seizure rate by 50% or higher;
b) reduction of the convulsive seizure frequency;
c) increased likelihood of being free from seizure;
d) longest seizure free interval;
e) number of seizure free days;
f) improvement assessed by Clinical Global Impression-Improvement;
g) improvement assessed by Patient's Global Impression of Change (PGI-C);
h) improvement assessed by Quality of Life in Childhood Epilepsy (QOLCE);
i) improvement assessed by Overall Quality of Life Score from the Pediatric Quality of Life Inventory™ (PedsQL) Score;
j) improvement assessed by Total Score from PedsQL Family Impact Module Score;
k) improvement assessed by Quality of Life (QoL) of the Parent/Caregiver using the EQ-5D-5L Scale;
l) improvement assessed by Hospital Anxiety and Depression Scale (HADS); or
m) improvement on behavioral or cognitive symptoms.

Without further elaboration, it is believed that one skilled in the art can, based on the above description, utilize the present invention to its fullest extent. The following specific examples are therefore to be construed as merely illustrative, and not limitative of the remainder of the disclosure in any way whatsoever. All publications cited herein are incorporated by reference in their entirety.

### Pharmaceutical Compositions

In one aspect, a pharmaceutical composition comprising the compound of Formula I, or a pharmaceutically acceptable salt thereof, and a pharmaceutically acceptable excipient, may be administered for the treatment of DVS in a subject in need thereof. In various embodiments, the composition is a solid pharmaceutical composition.

The pharmaceutical compositions provided herein can be administered by a variety of routes including, but not limited to, oral administration, administration as a suppository, topical contact, parenteral administration (e.g., intravenous, intramuscular, intra-arterial, intradermal, subcutaneous, intraperitoneal, intraventricular, and intracranial), intralesional administration, intrathecal administration, intranasal administration, transmucosal administration (e.g., buccal, sublingual, nasal, or transdermal), or the implantation of a slow-release device, e.g., a mini-osmotic pump, to a subject. In certain embodiments, the pharmaceutical compositions disclosed herein are administered orally.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. Unless defined otherwise, all abbreviations used herein have their conventional meaning within the chemical and biological arts. The chemical structures and formulae set forth herein are constructed according to the standard rules of chemical valency known in the chemical arts.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from the group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “comprise,” “comprises,” “comprising,” “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “composition” or “pharmaceutical composition” or “pharmaceutical formulation” refers to the combination of an active agent with an excipient or a carrier, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or a human, as appropriate, and/or that are approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., the compound of Formula I), which salt is compatible with pharmaceutical administration.

Examples of acids include, but are not limited to, hydrochloric, hydrobromic, sulfuric, nitric, perchloric, fumaric, malcic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, monosulfate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the subject is a participant. In some embodiments, the subject is a patient.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration, transmucosal administration (e.g., buccal, sublingual, nasal, or transdermal), or subcutaneous administration, or the implantation of a slow-release device, e.g., a mini-osmotic pump, to a subject. Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc.

By “co-administer,” it is meant that a composition described herein is administered at the same time, just prior to, or just after the administration of one or more additional therapies (e.g., anti-cancer agent, chemotherapeutic, or treatment for a neurodegenerative disease). The compound of formula I, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, and unless otherwise specified, the terms “treat,” “treating” and “treatment” include an action that occurs while a subject is suffering from the specified disease, disorder or condition, which reduces the severity of the disease, disorder or condition, or retards or slows the progression of the disease, disorder or condition (e.g., “therapeutic treatment”). “Treat,” “treating” and “treatment”, as used herein, can include any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The phrase “therapeutically effective amount,” as used herein, refers to the amount of a compound (e.g., a compound of Formula I), or a pharmaceutically acceptable salt thereof, that will elicit the biological or medical response of a tissue, system, animal or human that is being sought by the researcher, veterinarian, medical doctor or other clinician. The compound, or a pharmaceutically acceptable salt thereof, described in the present disclosure can be administered in therapeutically effective amounts to treat a disease. A therapeutically effective amount of a compound, or a pharmaceutically acceptable salt thereof, can be the quantity required to achieve a desired therapeutic and/or prophylactic effect, such as an amount which results in lessening of a symptom of a disease such as DVS.

### EXAMPLES

In order that the disclosure described herein may be more fully understood, the following examples are set forth. The examples described in this application are offered to illustrate the compound, pharmaceutical compositions, and methods described herein and are not to be construed in any way as limiting their scope.

### Example 1: Synthesis of 2-chloro-4-[1-(4-fluorophenyl)-2,5-dimethyl-1H-imidazol-4-ylethynyl]pyridine (Compound of Formula I, supra; See U.S. Pat. No. 7,332,510).

2-Chloro-4-[1-(4-fluorophenyl)-2-methyl-1H-imidazol-4-ylethynyl]-pyridine (200 mg, 0.6 mmol) was dissolved in 10 mL tetrahydrofuran (THF) and cooled to −75° C. Lithiumdiisopropylamide (0.45 mL, 0.91 mmol) was added and the mixture stirred for 15 min at −75° C. Iodomethane (0.05 mL, 0.85 mmol) was added and stirring was continued at −75° C. for 2 hrs. The reaction mixture was quenched with saturated NaHCO₃ solution and extracted with water and ethyl acetate. The combined organic extracts were dried with sodium sulfate, filtered and evaporated. The crude product was purified by flash chromatography on silica gel (heptane/ethyl acetate 90:10 to 20:80 gradient) and by recrystallization from ethyl acetate. The title compound was obtained as a white solid. MS: m/z=326.5 (M+H+).

### Example 2: Preparation of Polymorphs of Salts of Compound of Formula I (See U.S. Pat. No. 8,063,076).

Form A monosulfate salt: 61.0 g of 2-chloro-4-[1-(4-fluoro-phenyl)-2,5-dimethyl-1H-imidazol-4-ylethynyl]-pyridine was dissolved in 610 mL of 2-propanol. The solution was filtered and the filter rinsed with 31 mL of 2-propanol. To the combined solutions a mixture of 30 mL of water and 18.91 g of sulfuric acid (97%) was added drop-wise. The solution was cooled to 0-5° C. Seeding was performed at 58° C. as needed. The solid residues were filtered, washed with 2-propanol (0-5° C.) and dried at 50° C. and less than 1 mbar for 18 hrs to provide the monosulfate salt of the compound of Formula I in a yield of 69.1 g (87.1%). Form A seeding crystals can be prepared upon cooling crystallization of a hot solution of 250 mg of the monosulfate salt in 10 mL of 2-propanol. After cooling to 0° C., the solid residues can be filtered and dried at 50° C. under vacuum to afford Form A monosulfate salt, which was confirmed by the XRPD pattern as substantially shown in FIG. 1.

Form B monosulfate salt: 300 mg of Form A monosulfate salt of the compound of Formula I was dissolved in 3 mL 2-propanol and 1 mL water at 60° C. to produce a clear solution. The clear solution was seeded with Form B monosulfate salt and sealed at room temperature (e.g., about 25° C.). Single crystals were formed after 3 days. Seeding crystals can be prepared by formation of a saturated slurry of Form A monosulfate salt of the compound of Formula I in 2-propanol and water (3:1 v/v) at room temperature. The slurry was stirred at room temperature for approximately 3 weeks. The solids were filtered via a glass 35 filter to afford crystalline Form B monosulfate salt, which was confirmed by the XRPD pattern as substantially shown in FIG. 2.

Form C hemisulfate salt: 41 g of Form A monosulfate of the compound of Formula I was mixed with 128 g of water. The slurry was stirred at room temperature for 2-16 hrs. After all the Form A monosulfate salt had been converted to the hemisulfate salt, the resulting crystals were collected by filtration and rinsed with water. The wet cake thus obtained was dried at 40° C. in a vacuum oven for 48 hrs to afford Form C hemisulfate salt in a yield of 93%. Form Chemisulfate salt was confirmed by the XRPD pattern as substantially shown in FIG. 3.

Amorphous monosulfate salt: 0.53 g of a monosulfate of the compound of Formula I was dissolved in 10 mL of methanol at approximately 65° C. After complete evaporation of the solvent under vacuum, the solid (foam) was further dried at about 50° C. under 5-20 mbar for 18 hrs. Analysis (XRPD and DSC) revealed amorphous form of the compound of Formula I was obtained. The amorphous monosulfate salt was characterized by an infrared spectrum having bands at 2730, 2592, 2219, 1633, 1586, 1570, 1513, 1375, 1343, 1293, 1226, 1157, 1130, 1084, 1040, 986, 903, 848, 788, 712 and 670 cm⁻¹ (±3 cm⁻¹). The glass transition temperature (T_{g}) of the amorphous form determined by DSC was largely dependent on the solvent content and was observed for the wet sample (closed pan) at about 42° C. and for the in-situ dried sample (pan with perforation lid) at about 77° C.

### Example 3: A Study of the Compound of Formula I for Treatment of Subjects With Dravet Syndrome (DVS)

A Phase 2, multicenter, 12-week at maintenance dose, prospective, parallel group, double-blind randomized, placebo-controlled study would be carried out to evaluate the efficacy and safety of daily 0.5 to 3.5 mg of the compound of Formula I (referred to herein as “CF-I” or “Compound I”) adjunctive to ongoing seizure therapy in patients with Dravet Syndrome (DVS) with suboptimal response to their current anti-seizure medication.

### A. Primary Outcome Objectives

To evaluate:
1. Change From Baseline in the Mean Convulsive Seizures Frequency (MCSF) to the Combined Titration and Maintenance Periods (T+M) in Participants Receiving Compound I 0.5 to 3.5 mg Compared to Placebo [Time Frame: From Baseline approximately 18 weeks [Titration Period (6 weeks) plus Maintenance Period (12 weeks)]]
2. Change in mean convulsive seizure frequency comparing the baseline with the combined titration and maintenance period for Compound I 0.5 to 3.5 mg group compared with placebo group.

### B. Secondary Outcome Objectives

To evaluate:
1. Change From Baseline in the Mean Convulsive Seizures Frequency to the Combined Titration and Maintenance Period (T+M) in Participants Receiving Compound I 0.5 to 3.5 mg compared to Placebo [Time Frame: From Baseline approximately 18 weeks [Titration Period (6 weeks) plus Maintenance Period (12 weeks)]] Convulsive seizures include hemiclonic, focal with clear observable motor signs, generalized tonic clonic, secondarily generalized tonic clonic, tonic, clonic, and drop seizures (tonic/atonic).
2. Percentage of Participants Who Achieved Greater Than or Equal to 25% (≥25%) Reduction in Convulsive Seizure Frequency in Each Compound I Treatment Arm Compared to Placebo From Baseline During the Titration and Maintenance Period [Time Frame: From Baseline approximately 18 weeks [Titration Period (6 weeks) plus Maintenance Period (12 weeks)]] Convulsive seizures include hemiclonic, focal with clear observable motor signs, generalized tonic clonic, secondarily generalized tonic clonic, tonic, clonic, and drop seizures (tonic/atonic).

3. Percentage of Participants Who Achieved a ≥50% Reduction in Convulsive Seizure Frequency in Each Compound I Treatment Arm Compared to Placebo From Baseline During the Titration and Maintenance Period [Time Frame: From Baseline approximately 18 weeks [Titration Period (6 weeks) plus Maintenance Period (12 weeks)]] Convulsive seizures include hemiclonic, focal with clear observable motor signs, generalized tonic clonic, secondarily generalized tonic clonic, tonic, clonic, and drop seizures (tonic/atonic).
4. Percentage of Participants Who Achieved a ≥75% Reduction in Convulsive Seizure Frequency in Each Compound I Treatment Arm Compared to Placebo From Baseline During the Titration and Maintenance Period [Time Frame: From Baseline approximately 18 weeks [Titration Period (6 weeks) plus Maintenance Period (12 weeks)]] Convulsive seizures include hemiclonic, focal with clear observable motor signs, generalized tonic clonic, secondarily generalized tonic clonic, tonic, clonic, and drop seizures (tonic/atonic).
5. Percentage of Participants Who Achieved a 100% Reduction in Convulsive Seizure Frequency in Each Compound I Treatment Arm Compared to Placebo From Baseline During the Titration and Maintenance Period [Time Frame: From Baseline approximately 18 weeks [Titration Period (6 weeks) plus Maintenance Period (12 weeks)]] Convulsive seizures include hemiclonic, focal with clear observable motor signs, generalized tonic clonic, secondarily generalized tonic clonic, tonic, clonic, and drop seizures (tonic/atonic).
6. Longest Convulsive Seizure-free Interval in Each Compound I Treatment Arm Compared to Placebo During the Titration and Maintenance Period [Time Frame: From Baseline approximately 18 weeks [Titration Period (6 weeks) plus Maintenance Period (12 weeks)]] The longest interval between convulsive seizures is calculated over the entire Titration and Maintenance Period and is derived as the maximum of the number of days between consecutive convulsive seizures.
7. Number of Convulsive Seizure-free Days in Each Compound I Treatment Arm Compared to Placebo During the Titration and Maintenance Period [Time Frame: From Baseline approximately 18 weeks [Titration Period (6 weeks) plus Maintenance Period (12 weeks)]] A convulsive seizure free day is defined as a day for which no convulsive seizures is reported.
8. Change From Baseline in Non-convulsive Seizure Frequency to the Combined Titration and Maintenance Period in Each Compound I Treatment Arm Compared to Placebo [Time Frame: From Baseline approximately 18 weeks [Titration Period (6 weeks) plus Maintenance Period (12 weeks)]] Non-convulsive seizures include focal without clear observable motor signs, absence or atypical absence, myoclonic and atonic.
9. Change From Baseline in Convulsive+Non-convulsive Seizure Frequency to the Combined Titration and Maintenance Period in Each Compound I Treatment Arm Compared to Placebo [Time Frame: From Baseline approximately 18 weeks [Titration Period (6 weeks) plus Maintenance Period (12 weeks)]] Total seizure frequency is defined as the combination of convulsive and non-convulsive seizures. Convulsive seizures include hemiclonic, focal with clear observable motor signs, generalized tonic clonic, secondarily generalized tonic clonic, tonic, clonic, and drop seizures (tonic/atonic). Non-convulsive seizures include focal without clear observable motor signs, absence or atypical absence, myoclonic and atonic.
10. Percentage of Participants With Rescue Medication Usage in Each Compound I Treatment Arm Compared to Placebo During the Titration and Maintenance Period [Time Frame: From Baseline approximately 18 weeks [Titration Period (6 weeks) plus Maintenance Period (12 weeks)]] Rescue medication is administered according to each participant's usual or prescribed regimen consisting of 1 or more medications.
11. Percentage of Participants With Hospitalization and Healthcare Resource Utilization to Treat Seizures in Each Compound I Treatment Arm Compared to Placebo During [Time Frame: From Baseline approximately 18 weeks [Titration Period (6 weeks) plus Maintenance Period (12 weeks)]] An exemplary participant utilizes a medical center care to treat a seizure during the study.
12. Percentage of Participants With Status Epilepticus (SE) in Each Compound I Treatment Arm Compared to Placebo During the Titration and Maintenance Period [Time Frame: From Baseline approximately 18 weeks [Titration Period (6 weeks) plus Maintenance Period (12 weeks)]] An exemplary participant has SE episode recorded as an adverse event (AE) during treatment or a seizure greater than 10 minutes, for each treatment group.
13. Distribution of Duration of Convulsive Seizures (in Percentage) in Each Compound I Treatment Arm Compared to Placebo at Baseline and During the Titration and Maintenance Period [Time Frame: From Baseline approximately 18 weeks [Titration Period (6 weeks) plus Maintenance Period (12 weeks)]]
14. Percentage of Participants With Clinical Global Impression-Improvement (CGI-I) Rating Score, as Assessed by the Principal Investigator in Each Compound I Treatment Arm Compared to Placebo. CGI-I scale measures improvement in the participant's clinical status from Baseline. The severity of a participant's condition can be rated on a 7-point scale ranging from 1 (very much improved) to 7 (very much worse), as follows: 1—very much improved, 2—much improved, 3—minimally improved, 4—no change, 5—minimally worse, 6—much worse and 7—very much worse. The Principal Investigator may rate the global impression of the participant's condition during a study.

15. Percentage of Participants With Clinical Global Impression-Improvement Rating Score, as Assessed by the Parent/Caregiver in Each Compound I Treatment Arm Compared to Placebo. CGI-I scale measures improvement in the participant's clinical status from Baseline. The severity of a participant's condition can be rated on a 7-point scale ranging from 1 (very much improved) to 7 (very much worse), as follows: 1—very much improved, 2—much improved, 3—minimally improved, 4—no change, 5—minimally worse, 6—much worse and 7—very much worse. The Parent/Caregiver can rate the global impression of the participant's condition during a study.
16. Change From Baseline in the Quality of Life in Childhood Epilepsy (QOLCE) Score to Measure Quality of Life in Each Compound I Treatment Arm Compared to Placebo. QOLCE is a low-burden parent/caregiver completed assessment that evaluates how epilepsy affects day-to day functioning of the participant in various life areas, including physical activities, well-being, cognition, social activities, behavior, and general health. QOLCE scores items on 16 subscales with possible 5-point response for each, where scores of 5 was best possible response and 1 was worst possible response. Item scores are then transformed to a 0-100 scale as follows: 1-0, 2-25, 3-50, 4-75, 5-100. A score for each participant for each subscale can be calculated by averaging that participant's responses to each item in the subscale. Subscale scores per participant can be averaged to obtain an overall QoL score for each participant. Higher the subscale and overall QOL scores, better the response.
17. Change From Baseline in the Overall Quality of Life Score From the Pediatric Quality of Life Inventory™ (PedsQL) Score in Each Compound I Treatment Arm Compared to Placebo. The Pediatric Quality of Life Inventory (PedsQL) is a pediatric modular measure of health-related quality of life (QoL) completed by the parent/caregiver on behalf of the participant. It consisted of 23 items across 4 core scales that measure physical (8 items), emotional, social, and school functioning (5 items each). Each of the responses to the 23 items is initially scored on a 5-point Likert scale from 0 (Never) to 4 (Almost always). Scores are linearly transformed to a scale of 0 to 100, where 0=100, 1=75, 2=50, 3=25 and 4=0, and higher scores correspond to better health-related QoL. The Overall Quality of Life is the average of all the items over the number of items answered on all the Scales.
18. Change From Baseline in the Total Score From PedsQL Family Impact Module Score in Each Compound I Treatment Arm Compared to Placebo. The PedsQL Family Impact measures the impact of pediatric chronic health conditions on parents and the family by measuring parent self-reported physical, emotional, social, and cognitive functioning, communication, worry, and family daily activities and relationships. There are a total of 36 items in the PedsQL: 6 items for Physical Functioning, 5 items each for Emotional Functioning, Cognitive Functioning and Worry, 4 for Social Functioning, 3 for Communication, 3 questions for Daily Activities, and 5 for Family Relationships. Each of the responses are initially scored on a 5-point Likert scale from 0 (Never) to 4 (Almost always) and then linearly transformed to a scale of 0 to 100, where 0=100, 1=75, 2=50, 3=25 and 4=0, and higher scores mean better health-related QoL.
19. Quality of Life (QoL) of the Parent/Caregiver Using the EQ-5D-5L Scale in Each Compound I Treatment Arm Compared to Placebo at Baseline and Day. The EuroQOL-5 Dimensions-5 Levels scale produced by European QOL Group (EQ-5D-5L) health questionnaire is a health-related QOL instrument with 5 dimensions: mobility, self-care, usual activities, pain/discomfort, and anxiety/depression. The 5 dimensions of EQ-5D-5L health questionnaire are assessed on a Likert scale with 5 possible levels: no problems, slight problems, moderate problems, severe problems, and extreme problems. The categories “slight problems”, “moderate problems”, “severe problems” and “extreme problems” are collapsed into one response category “problems.” The QOL of the parent/caregiver is assessed and percentage of participants is reported for each item.
20. Change From Baseline in Affective Symptoms of the Parent/Caregiver Using the Hospital Anxiety and Depression Scale (HADS) in Each Compound I Treatment Arm Compared to Placebo. The HADS is a tool that is validated to assess presence of anxiety or depression in an outpatient non-psychiatric population. HADS is a 14-item scale that generates ordinal data for 2 dimensions: 1) Anxiety (7 items), and 2) Depression (7 items). Each item has 4 possible answers rated 0 to 3, of which 0=No distress and 3=worst distress. All answers to the items for a dimension with their respective rating are added resulting in a range for each dimension from 0-21, out of which of 0-7=normal; 8-10=borderline abnormal; 11-21=abnormal. Scores for the entire scale (emotional distress) range from 0 to 42, with higher scores indicating more distress.
21. Proportion of subjects in each Compound I treatment arm compared with placebo who were considered treatment responders, defined as those with a ≥40% and ≥50%, reduction in convulsive seizures from baseline.
22. Comparison of subjects' longest seizure-free interval in each Compound I treatment arm compared with placebo. Comparison of each subjects' longest convulsive seizure-free interval, and longest interval without any seizures, during the 18-week titration+maintenance period, are calculated independently for the Compound I 0.5 to 3.5 mg treatment groups versus placebo.

### C. Additional Secondary Outcome Measures [Time Frame: Baseline Compared With Titration+Maintenance Period, or T+M as Appropriate]

To evaluate:
i. The number of convulsive seizure-free days.
ii. The proportion of subjects who achieve ≥75% reductions from baseline in convulsive seizure frequency.
iii. The change from baseline in non-convulsive seizure frequency and all seizure frequency.
iv. The incidence of rescue medication usage, and medical utilization.
v. The incidence of status epilepticus.
vi. Clinical Global Impression-Improvement rating, as assessed by the parent/caregiver.
vii. The change from baseline in Quality of Life.
viii. The change from baseline in affective symptoms of the parent/caregiver

### D. Safety and Tolerability

To study on safety and tolerability of Compound I 0.5 to 3.5 mg compared to placebo. Compare safety and tolerability of Compound I 0.5 to 3.5 mg/day and placebo with regard to adverse events (AEs), laboratory parameters, physical examination, neurological examination, vital signs (blood pressure, heart rate, temperature, and respiratory rate), electrocardiograms (ECG), echocardiograms (ECHO), body weight and cognitive function.

### E. Study Design

This is a multi-center, randomized, double-blind, parallel, placebo-controlled study in patients who, despite of an optimal treatment with antiseizure medications, show evidence of at least one type of generalized (convulsive) seizure, including drop seizures (atonic, tonic, tonic-clonic or myoclonic) for at least six months

Patients, who are treated with therapeutic doses of antiseizure medications for at least 6 months, sign the informed consent or have a legally authorized representative sign the informed consent, meet the study eligibility criteria, and agree to participate in the study or have a legally authorized representative to agree for the patient to participate in the study, will enter an up to 8-week screening and stabilization period.

Eligible patients will be maintained on their antiseizure medication (same dose) and will be randomized via interactive voice/web response system to receive one of two treatments in addition to their background antiseizure medication.

Treatment A: Compound I 0.5 to 3.5 mg QD orally (note: QD refers to once daily)

Treatment B: Placebo QD orally

At the end of the stabilization period, patients who continue to meet all entry criteria will be randomized to receive double blind treatment.

### F. Study Phases

The total double-blind study duration is approximately 18-weeks

Screening and stabilization Phase: up to 8 weeks

Treatment Phase: To include a titration phase of up to 6-weeks followed by 12-weeks maintenance phase

### G. Target Population

Patients must meet the following criteria for study entry:
1. Ability and willingness to provide written informed consent and to comply with the study procedures, or have a legally authorized representative that has the ability and willingness to provide written informed consent and to comply with the study procedures for the patient-infants to adults. A documented history of DVS. This includes DNA-confirmed and evidence of at least one type of generalized seizure, including convulsive seizures (atonic, tonic, tonic-clonic or myoclonic) for at least six months.
2. Subjects should be refractory; that is having documented failures on more than one antiseizure medication (ASM).
3. Subjects must be taking one or more ASMs at a dose, which has been stable for at least four weeks prior to screening.
4. All medications or interventions for seizures (including ketogenic diet and neurostimulator devices) must have been stable for four weeks prior to screening and patient is willing to maintain a stable regimen throughout the study. The ketogenic diet and neurostimulator deives are not accounted as an ASM.

Patients who meet any of the following criteria will be excluded from study participation:
1. Etiology of subject's seizures is a progressive neurologic disease.
2. Subject has had an anoxic episode requiring resuscitation within six months of screening.
3. Subject has clinically significant unstable medical conditions other than epilepsy.
4. Subject has had clinically relevant symptoms or a clinically significant illness in the four weeks prior to screening or randomization, other than epilepsy.
5. Subject has been part of a clinical trial involving another IMP in the previous six months.
6. Active suicidal behavior or any suicidal ideation of type four or five on the Columbia Suicide Severity Rating Scale in the last month or at screening.
7. Subject is currently taking long-term systemic steroids (excluding inhaled medication for asthma treatment) or any other daily medication known to exacerbate epilepsy. An exception will be made of prophylactic medication, for example, idiopathic nephrotic syndrome or asthma.
8. Subject is taking felbamate, and they have been taking it for less than one year prior to screening.
9. Concurrent use of fenfluramine. Participants with prior use of fenfluramine within the previous 3 months, or without proper documentation of an echocardiogram, at minimum 3 months following the last dose of fenfluramine, to ensure that the participant does not meet any criteria for drug-related (fenfluramine) cardiac valvular heart disease and/or drug-related pulmonary arterial hypertension (PAH)
10. Pregnancy or lactation

### H. Primary Outcome Measures (Efficacy)

The primary outcome measure is:
1. The change From Baseline in the Mean Convulsive Seizures Frequency (MCSF) to the Combined Titration and Maintenance Periods (T+M) in Participants Receiving Compound I 0.5 to 3.5 mg Compared to Placebo [Time Frame: From Baseline approximately 18 weeks [Titration Period (6 weeks) plus Maintenance Period (12 weeks)]]
2. The change in mean convulsive seizure frequency comparing the baseline with the combined titration and maintenance period for Compound 1 0.5 to 3.5 mg group compared with placebo group.

### I. Secondary Outcome Measures (Efficacy)

The secondary outcome measure is:
1. The change From Baseline in the Mean Convulsive Seizures Frequency to the Combined Titration and Maintenance Period (T+M) in Participants Receiving Compound I 0.5 to 3.5 mg compared to Placebo.
2. The percentage of Participants Who Achieved Greater Than or Equal to 25% (≥25%) Reduction in Convulsive Seizure Frequency in Each Compound I Treatment Arm Compared to Placebo From Baseline During the Titration and Maintenance Period.
3. The percentage of Participants Who Achieved a ≥50% Reduction in Convulsive Seizure Frequency in Each Compound I Treatment Arm Compared to Placebo From Baseline During the Titration and Maintenance Period.
4. The percentage of Participants Who Achieved a ≥75% Reduction in Convulsive Seizure Frequency in Each Compound I Treatment Arm Compared to Placebo From Baseline During the Titration and Maintenance Period.
5. The percentage of Participants Who Achieved a 100% Reduction in Convulsive Seizure Frequency in Each Compound I Treatment Arm Compared to Placebo From Baseline During the Titration and Maintenance Period.
6. The longest Convulsive Seizure-free Interval in Each Compound I Treatment Arm Compared to Placebo During the Titration and Maintenance Period.
7. The number of Convulsive Seizure-free Days in Each Compound I Treatment Arm Compared to Placebo During the Titration and Maintenance Period.
8. The change From Baseline in Non-convulsive Seizure Frequency to the Combined Titration and Maintenance Period in Each Compound I Treatment Arm Compared to Placebo.
9. The change From Baseline in Convulsive+Non-convulsive Seizure Frequency to the Combined Titration and Maintenance Period in Each Compound I Treatment Arm Compared to Placebo.
10. The percentage of Participants With Rescue Medication Usage in Each Compound I Treatment Arm Compared to Placebo During the Titration and Maintenance Period.
11. The percentage of Participants With Hospitalization and Healthcare Resource Utilization to Treat Seizures in Each Compound I Treatment Arm Compared to Placebo During.
12. The percentage of Participants With Status Epilepticus (SE) in Each Compound I Treatment Arm Compared to Placebo During the Titration and Maintenance Period.
13. The distribution of Duration of Convulsive Seizures (in Percentage) in Each Compound I Treatment Arm Compared to Placebo at Baseline and During the Titration and Maintenance Period.
14. The percentage of Participants With Clinical Global Impression-Improvement (CGI-I) Rating Score, as Assessed by the Principal Investigator in Each Compound I Treatment Arm Compared to Placebo.
15. The percentage of Participants With Clinical Global Impression-Improvement Rating Score, as Assessed by the Parent/Caregiver in Each Compound I Treatment Arm Compared to Placebo. CGI-I scale measures improvement in the participant's clinical status from Baseline.
16. The change From Baseline to Day 99 in the Quality of Life in Childhood Epilepsy (QOLCE) Score to Measure Quality of Life in Each Compound I Treatment Arm Compared to Placebo.
17. The change From Baseline to Day 99 in the Overall Quality of Life Score From the Pediatric Quality of Life Inventory™ (PedsQL) Score in Each Compound I Treatment Arm Compared to Placebo.
18. The change From Baseline to Day 99 in the Total Score From PedsQL Family Impact Module Score in Each Compound I Treatment Arm Compared to Placebo.
19. The quality of Life (QoL) of the Parent/Caregiver Using the EQ-5D-5L Scale in Each Compound I Treatment Arm Compared to Placebo at Baseline and Day.
20. The change From Baseline to Day 99 in Affective Symptoms of the Parent/Caregiver Using the Hospital Anxiety and Depression Scale (HADS) in Each Compound I Treatment Arm Compared to Placebo.
21. The proportion of subjects in each Compound I treatment arm compared with placebo who were considered treatment responders, defined as those with a ≥40% and ≥50%, reduction in convulsive seizures from baseline.
22. The comparison of subjects' longest seizure-free interval in each Compound I treatment arm compared with placebo.

### J. Additional Secondary Outcome Measures [Time Frame: Baseline Compared With Titration+Maintenance Period, or T+M as Appropriate]

The additional secondary outcome measure is:
ix. The number of convulsive seizure-free days.
x. The proportion of subjects who achieve ≥75% reductions from baseline in convulsive seizure frequency.
xi. The change from baseline in non-convulsive seizure frequency and all seizure frequency.
xii. The incidence of rescue medication usage, and medical utilization.
xiii. The incidence of status epilepticus.
xiv. The clinical Global Impression-Improvement rating, as assessed by the parent/caregiver.
xv. The change from baseline in Quality of Life.
xvi. The change from baseline in affective symptoms of the parent/caregiver

### Example 4: Seizure Freedom Study of Compound I

Dravet syndrome is a severe developmental and epileptic encephalopathy (DEE) most frequently caused by de novo pathogenic variants in SCN1A (the sodium voltage gated channel alpha subunit 1). In most Dravet cases, there is loss of function of SCN1A notably in interneurons, resulting in reduced ability to ameliorate electrical activity and a consequent electrical hyperexcitability and predisposition to seizures. Heterozygous deletion of Sca1a in mice (Scn1a+/−) recapitulates several core phenotypes of clinical Dravet syndrome, including temperature-dependent and spontaneous seizures, SUDEP, and behavioral abnormalities. Furthermore, Scn1a+/−mice exhibit a similar clinical response to standard antiseizure medications, including seizure exacerbation associated with lamotrigine, and seizure suppression with, for example clobazam and fenfluramine. As such, Scn1a+/−mice are a favourable model to enable translation of novel ASMs with varied mechanism of action to the clinic.

As used herein, “Compound I” refers to the compound of Formula I.

As used herein, “CTEP” refers to a compound having the formula C₁₉H₁₃ClF₃N₃O and the structure

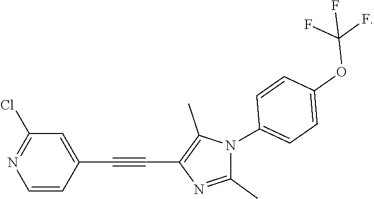 [SMILES: Cc1nc(C#Cc2ccnc(Cl)c2)c(C)n1-c1ccc(OC(F)(F)F)cc1]

The compound is known as a selective mGluR₅ allosteric antagonist or negative allosteric modulator.

As used herein, “RO6807794” refers to a compound having the formula C₁₈H₁₈FN₃O and the structure

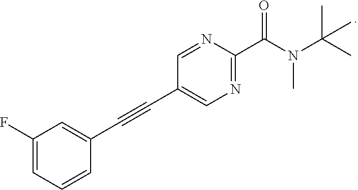 [SMILES: CN(C(=O)c1ncc(C#Cc2cccc(F)c2)cn1)C(C)(C)C]

The compound is known as a positive allosteric modulator.

As used herein, “NAM” refers to negative allosteric modulator, and “PAM” refers to positive allosteric modulator.

### A. Objective

The experiment was conducted to evaluate the effect of Compound I, CTEP and RO6807794 on seizure frequency, seizure freedom and survival in Scn1a+/− mice.

### B. Methods

Five treatment groups were studied to evaluate the effect of both negative and positive allosteric modulators of mGluR5 on seizure frequency, seizure freedom, and survival in Scn1a+/− mice:
(1) 0.3 mg/kg PO of Compound I, administered once daily for 28-days beginning on postnatal day 10 (P10).
(2) 1 mg/kg PO of Compound I, administered once daily for 28-days beginning on P10.
(3) 0.3 mg/kg PO of CTEP, administered every other day for 28-days beginning on P01.
(4) 0.3 mg/kg IP, RO6807794, administered once daily for 5-days beginning on P21.
(5) Untreated group, which did not receive dose administration.

Frontal cortex recordings were selected as representative of the seizure phenomenology on mice experiencing generalized seizures. EEG headmounts were implanted between P20-22 and recordings were initiated for continuous monitoring for a period of approximately 14-days for the NAM group and PAM group. Individual frontal cortex seizure measurements over time were visually explored and summaries across the time course were calculated.
1. Seizure frequency

Seizure frequency was measured by count of the number of seizures observed each day. The frequency was analyzed using a Chi-square test (all treatments) and Fisher's Exact test (pairwise treatment comparisons).
2. Seizure freedom

Seizure freedom was measured with a binary (0 or 1) endpoint, where it was recorded as a 0 if no seizure were observed through all study days, or 1 if at least 1 seizure was recorded across the whole study. Seizure freedom was summarized across all animals into a contingency table and analyzed using a generalized linear model (GLM) with a binomial distribution for the errors and a logit link function. The results were presented as probabilities and odds ratios between groups and 95% confidence intervals. The odd ratio described how much more likely it is for an animal to have no seizures (Seizure freedom=1) in a treated group compared to the untreated group. Analyses were performed using GraphPad Prism 9 (contingency table) and SAS 9.4 (binomial model).
3. Time to first seizure:

Time to first seizure refers the time recorded as the day on which the first seizure occurred (an observed, uncensored observation), or the final day of recording for that animal if on seizure was observed (a censored observation). Time to first seizure was visualized using a Kaplan-Meier plot and analyzed using a Log-rank (Mantel-Cox) test. Analyses were performed using GraphPad Prism 9.
4. Total seizure burden

Total seizure burden is sum of the seizure frequency across all study days.
5. Seizure per day

Seizure per day is measured by the total seizure count divided by the number of study days, wherein a seizure count measurement was adjusted by the number of days the animal was in the study.

Time to first seizure and Seizure per day were explored visually and analyzed using analysis of variance (ANOVA). Results were presented as geometric means, ratios of geometric means, confidence intervals and p-values. An additional non-parametric analysis, Kruskal-Wallis, was explored along with Wilcoxon pairwise comparisons to the Untreated group. The data was also analyzed using a generalized linear model (GLM) with a Poisson distribution for the errors and a log link function. The linear predictor described a model suitable for exploring a fixed effect of treatment group.

### C. Results 1. Seizure Freedom

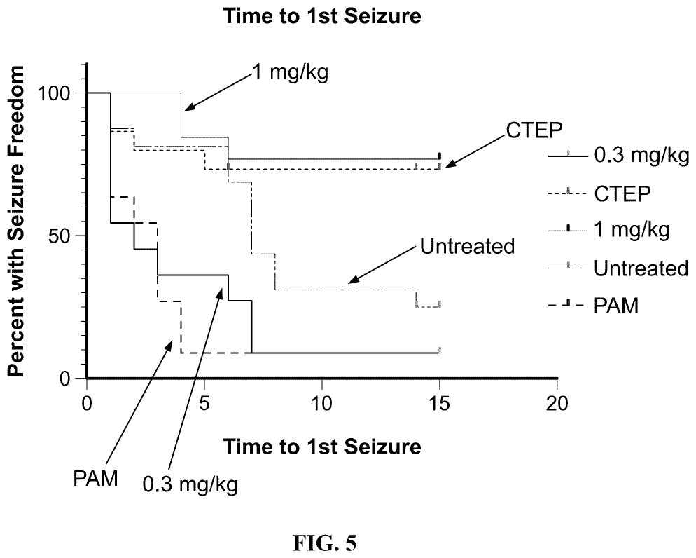

Seizure freedom was measured and analyzed for Compound I 1 mg/kg, Compound I 0.3 mg/kg, CTEP and Untreated groups. FIG. 4 and Table 4 provide the frequency and percent of animals exhibiting seizure freedom across four treatment groups. RO6807794 group was not included in the analysis of Seizure Freedom because it was dosed for only 5 days. FIG. 4 and Table 4 reveal that the percentage of animals exhibiting seizure freedom in the Compound I mg/kg is larger than the Untreated group. Compound I administration was associated with an increased probability of seizure freedom, with Compound I 1 mg/kg demonstrating a 77% chance of seizure freedom compared to 25% in the Untreated group (FIG. 4).

TABLE 1
Frequency and Percent of Animals Exhibiting Seizure Freedom

| Treatment group | At least 1 Seizure | No Seizures |
|---|---|---|
| Untreated | 12 (75%) | 4 (25%) |
| Compound I 0.3 mg/kg | 10 (90.9%) | 1 (9.1%) |
| Compound I 1 mg/kg | 3 (23.1%) | 10 (76.9%) |
| CTEP | 4 (26.7%) | 11 (73.3%) |
| Total | 29 (52.7%) | 26 (47.3%) |

As part of the contingency table analysis, a Chi-square test was performed on Table 4. The test returned a statistically significant p-value (p=0.0004) indicating that the proportion of animals with seizure freedom is dependent on the treatment group. The dependency on treatment group was demonstrated further in the GLM analysis, where the results were quantified in Table 5 through the probability of seizure freedom, odds ratios (vs Untreated) and 95% confidence intervals. As shown in Table 5, Compound I 1 mg/kg group is associated with an increased probability of seizure freedom and it is much more likely for an animal to have no seizures (Seizure freedom=1) in Compound I 1 mg/kg group compared to the Untreated group.

TABLE 2
Dependency of Seizure Freedom on Treatment Groups

| | 95% CI | | | | |
|---|---|---|---|---|---|
| Treatment group | Probability | Odds Ratio | Lower | Upper | p-value |
| Compound I 0.3 mg/kg | 0.09 | 0.3 | 0.03 | 3.32 | 0.32 |
| Compound I 1 mg/kg | 0.77 | 10 | 1.72 | 58.00 | 0.011 |
| CTEP | 0.73 | 8.25 | 1.59 | 42.89 | 0.013 |
| Untreated | 0.25 | | | | |

2. Time to First Seizure

For Compound I 1 mg/kg, Compound I 0.3 mg/kg, CTEP, RO6807794 and Untreated groups, a time-to-event type analysis (often called Survival analysis) was conducted with using the day on which each animal exhibited their first seizure. Each animal was defined as having Seizure freedom up until the day on which they exhibited their first seizure. If an animal was removed from the study early, or maintained Seizure freedom through day 15, they were classed as a censored observation, where the time to first seizure had not been observed during the time period of the study. It was understood that the time is greater than the final study day.

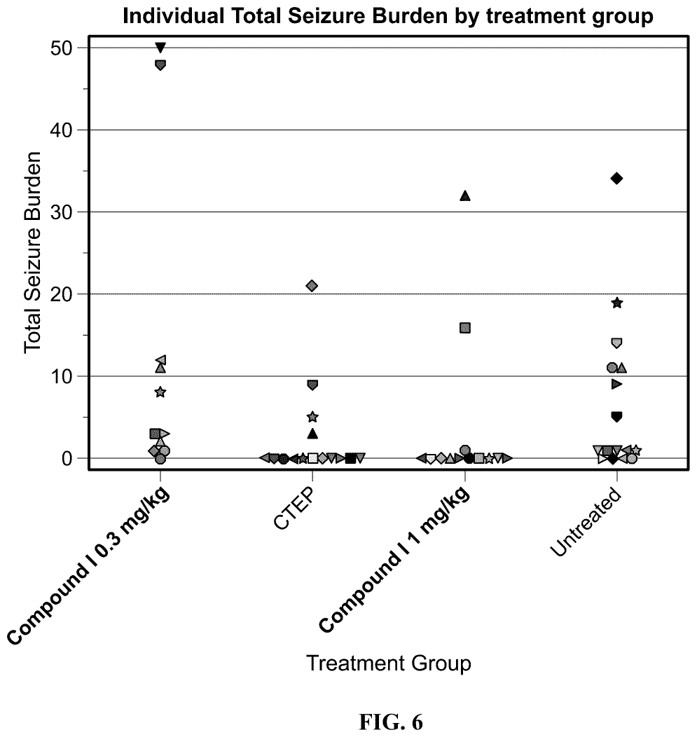

FIG. 5 illustrates a Kaplan-Meier plot that shows how the percent of animals with seizure freedom changes over time for all of the treatment groups. In FIG. 5, all groups start with 100% of animals exhibiting seizure freedom and the lines step down (the percent decreases) each time an animal in that group exhibits at least 1 seizure during that day. FIG. 5 demonstrates that Compound I 1 mg/kg and CTEP groups exhibit more animals with Seizure Freedom for a longer duration and this was quantified through the median time to first seizure. The median time to first seizure (or the time to 50% of the animals having at least 1 seizure) in the Compound I 0.3 mg/kg group was 2 days, the RO6807794 group was 3 days and the Untreated group was 7 days. By the end of the 15 days, Compound I 1 mg/kg had more than 50% of the animals maintaining Seizure freedom.

Compound I 1 mg/kg showed an increase in the time taken before at least one observed seizure, with 50% of the Untreated group experiencing at least one seizure by day 7, and 77% of the Compound I 1 mg/kg group remaining seizure free by day 15.
3. Total Seizure Burden

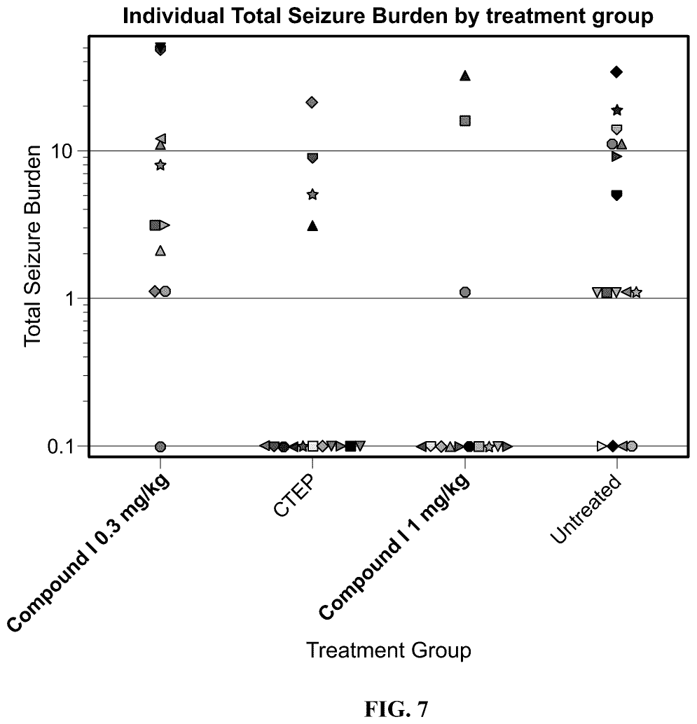

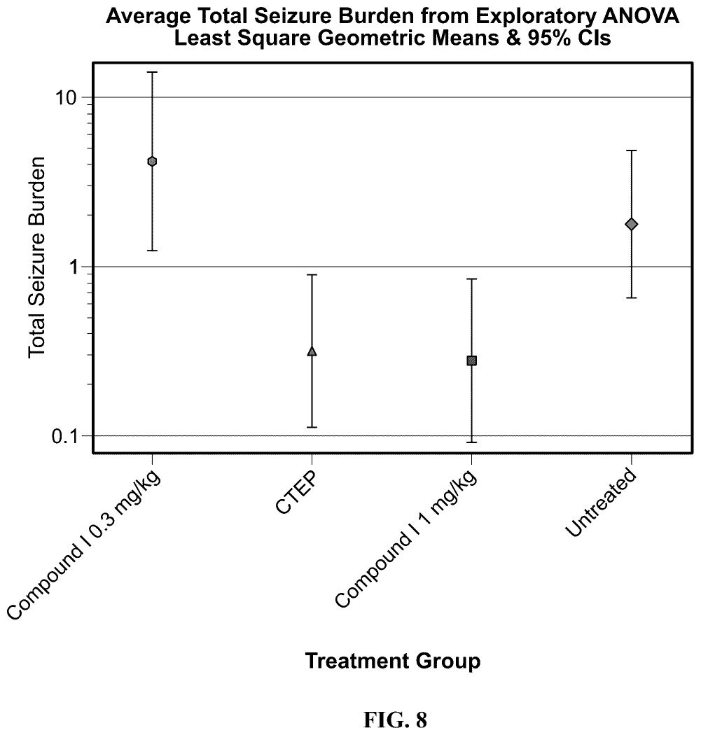

The sum of the seizure frequency across all study days were measured and analyzed for Compound I 1 mg/kg, Compound I 0.3 mg/kg, CTEP and Untreated groups. FIG. 6 illustrates a plot of the individual Total seizure burden data on the main treatment groups. FIG. 7 illustrates a plot of adjusted data of FIG. 6 using a logarithmic scale on the y axis. To generate the plot in FIG. 7, a small constant (10% of the smallest non-zero value, i.e. 0.1) was added to every response to visualize the data in FIG. 6 on log₁₀ transformed scale. RO6807794 group was not included in the analysis of Total Seizure Burden because it was dosed for only 5 days.

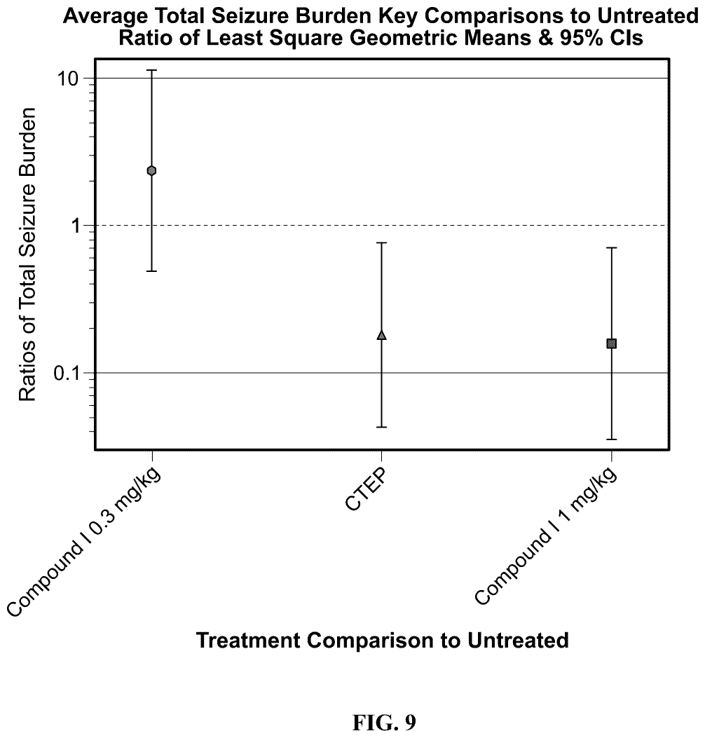

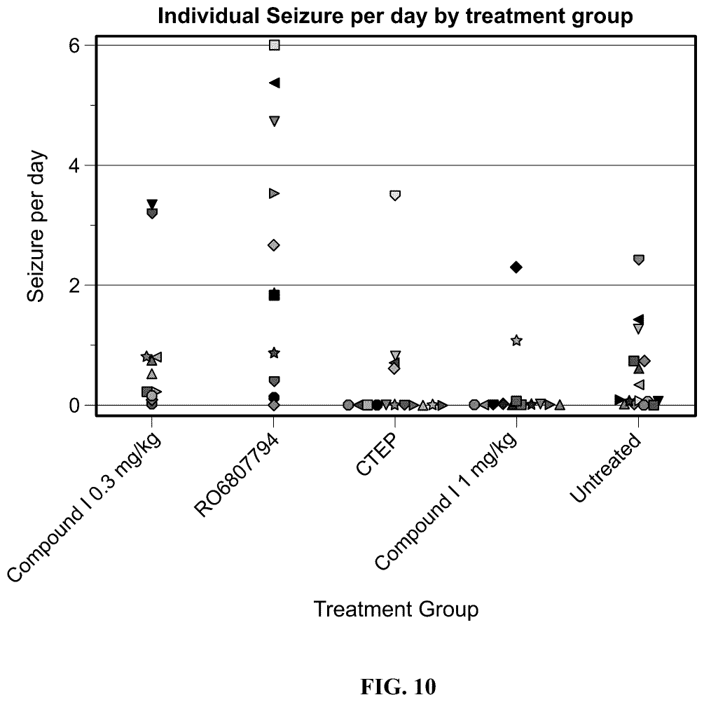

Exploratory ANOVA on log₁₀ transformed scale: An exploratory ANOVA was performed on the log transformed log data. The results are illustrated in FIG. 8 and FIG. 9. FIG. 8 contains the geometric means and 95% confidence intervals for the four treatment groups. FIG. 9 contains the comparisons against the Untreated group, expressed as ratios of geometric means and 95% confidence intervals. Comparisons where the confidence interval does not cross the reference line (Y=1) are considered statistically significant at the 5% significance level (p<0.05). Ratio is expressed as a percent change in Table 6. Both the CTEP and Compound I 1 mg/kg treatment groups exhibit statistically significantly lower total seizure burden compared to the Untreated group (reductions of 82% and 84% respectively, and p<0.05 for both). Compound I 1 mg/kg treatment groups exhibit statistically lower total seizure burden compared to both CTEP and the Untreated group.

TABLE 3
Geometric Means and 95% Confidence Intervals for Total Seizure Burden and Ratios of Means
and 95% Confidence Intervals for Total Seizure Burden compared to Untreated group

| | Geometric | Lower | Upper | Ratio of | Lower | Upper | Unadjusted | % |
|---|---|---|---|---|---|---|---|---|
| | Mean | 95% CI | 95% CI | Means | 95% CI | 95% CI | P value | Change |
| Untreated | 1.76 | 0.65 | 4.80 | | | | | |
| Compound I | 4.16 | 1.24 | 13.95 | 2.36 | 0.49 | 11.39 | 0.2777 | 136.21 |
| 0.3 mg/kg | | | | | | | | |
| Compound I | 0.28 | 0.09 | 0.84 | 0.16 | 0.04 | 0.71 | 0.0167 | −84.25 |
| 1 mg/kg | | | | | | | | |
| CTEP | 0.32 | 0.11 | 0.89 | 0.18 | 0.04 | 0.76 | 0.0205 | −82.08 |

Non-Parametric Analysis: A Kruskal-Wallis analysis with Wilcoxon pairwise tests were performed to explore the total seizure burden without assuming a specific underlying distribution. Like the exploratory ANOVA, these methods also indicated that the Compound I 1 mg/kg and CTEP groups exhibited lower total seizure burden compared to the Untreated group (p<0.05 for both).

GLM suitable for Discrete data: The GLM analysis performed was suitable for discrete (count) data and is presented in Table 7, using geometric means and 95% confidence intervals for the four treatment groups and comparisons against the Untreated group, expressed as ratios of geometric means and 95% confidence intervals. Both the CTEP and Compound I 1 mg/kg treatment groups exhibit statistically significantly lower total seizure burden compared to the Untreated group (reductions of 62% and 44% respectively, and p<0.01 for both).

### Table 4 Generalized Linear Madel Analveic an Discrete (Count) Data for Total Seizure

TABLE 4
Generalized Linear Model analysis on Discrete (count) Data for Total Seizure Burden

| | Geometric | Lower | Upper | Ratio of | Lower | Upper | Unadjusted | % |
|---|---|---|---|---|---|---|---|---|
| | Mean | 95% CI | 95% CI | Means | 95% CI | 95% CI | P value | Change |
| Compound I | 12.64 | 10.66 | 14.98 | 1.87 | 1.45 | 2.42 | <.0001 | 87.21 |
| 0.3 mg/kg | | | | | | | | |
| CTEP | 2.53 | 1.83 | 3.51 | 0.38 | 0.26 | 0.55 | <.0001 | −62.47 |
| Compound I | 3.77 | 2.83 | 5.02 | 0.56 | 0.4 | 0.79 | 0.0014 | −44.16 |
| 1 mg/kg | | | | | | | | |
| Untreated | 6.75 | 5.56 | 8.19 | | | | | |

4. Seizure per day

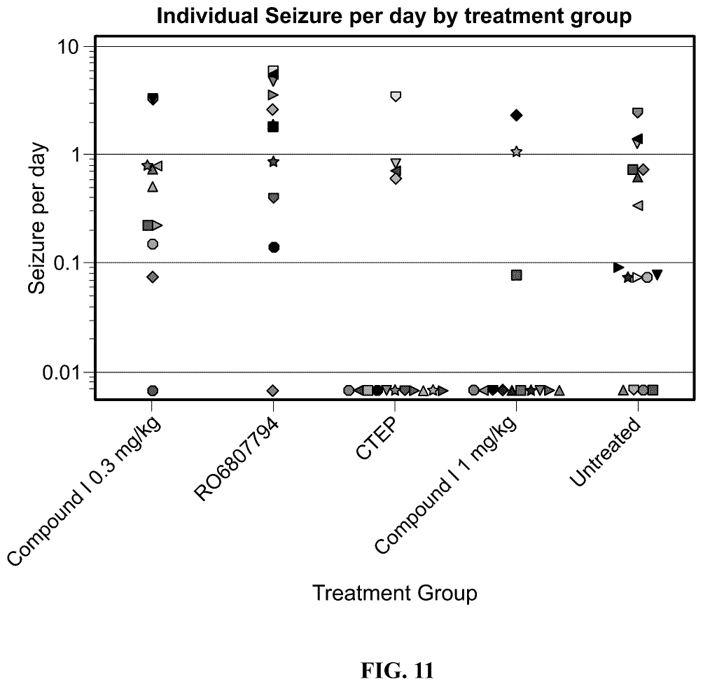

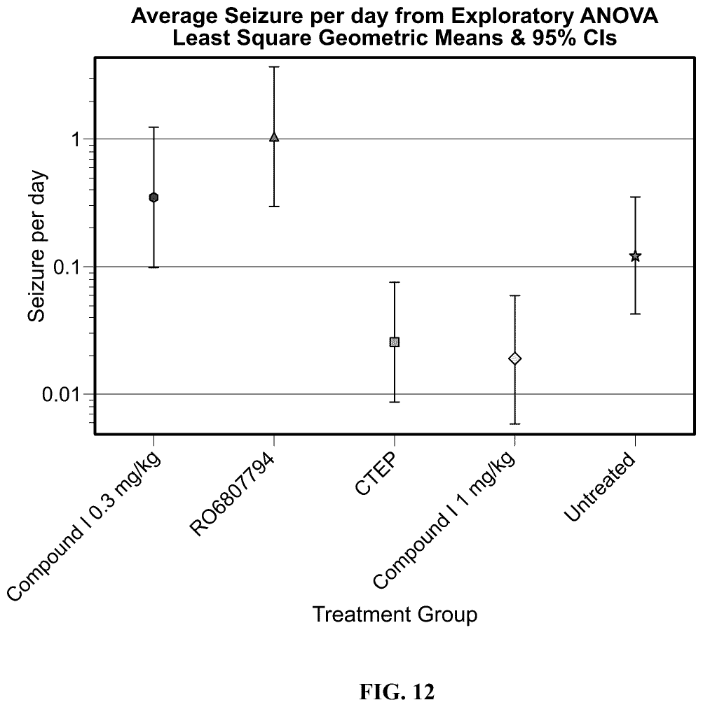

The total seizure burden measurement was adjusted for the number of days each animal was present in the study by a simple division. The individual seizure per day data are illustrated in FIG. 10 for all treatment groups. To enable the data to be visualized on the log10 transformed scale, a small constant (10% of the smallest non-zero value, i.e. 0.0067) was added to every response. FIG. 11 shows the adjusted data using a logarithmic scale on the y axis.

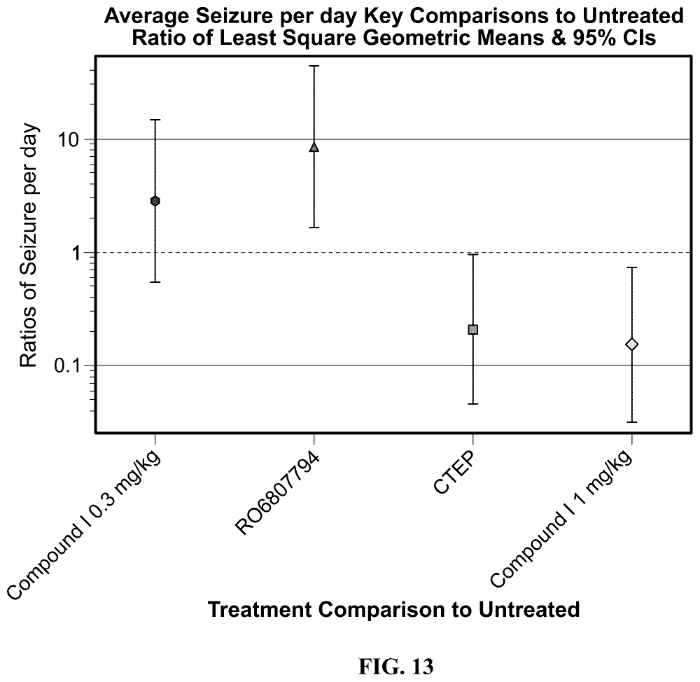

Exploratory ANOVA on log₁₀ transformed scale: An ANOVA was performed on the log transformed adjusted data. The results are shown in FIG. 12, FIG. 13, and Table 8. FIG. 12 plot contains the geometric means and 95% confidence intervals for the four treatment groups and FIG. 13 contains the comparisons against the Untreated group, expressed as ratios of geometric means and 95% confidence intervals. Comparisons where the confidence interval does not cross the reference line (Y=1) are considered statistically significant at the 5% significance level (p<0.05). The ratio is expressed as a percent change in Table 8. Both the CTEP and Compound I 1 mg/kg treatment groups exhibit statistically significantly lower average seizure per day compared to the Untreated group (reductions of 85% and 79% respectively, and p<0.05 for both, ANOVA on log₁₀ adjusted scale). Compound I 1 mg/kg treatment groups exhibit statistically lower average seizure per day compared to the Untreated group.

TABLE 5
Ratios of Geometric Means and 95% Confidence Intervals for Seizure per day and ratios
of means and 95% confidence intervals for seizures per day compared to untreated group

| | Geometric | Lower | Upper | Ratio of | Lower | Upper | Unadjusted | % |
|---|---|---|---|---|---|---|---|---|
| | Mean | 95% CI | 95% CI | Means | 95% CI | 95% CI | P value | Change |
| Compound I | 0.349 | 0.099 | 1.234 | 2.84 | 0.55 | 14.66 | 0.208 | 183.96 |
| 0.3 mg/kg | | | | | | | | |
| RO6807794 | 1.046 | 0.296 | 3.699 | 8.51 | 1.65 | 43.93 | 0.011 | 751.1 |
| CTEP | 0.026 | 0.009 | 0.076 | 0.21 | 0.05 | 0.94 | 0.042 | −79.05 |
| Compound I | 0.019 | 0.006 | 0.060 | 0.15 | 0.03 | 0.73 | 0.019 | −84.81 |
| 1 mg/kg | | | | | | | | |
| Untreated | 0.123 | 0.043 | 0.35 | | | | | |

Non-Parametric Analysis: A Kruskal-Wallis analysis with Wilcoxon pairwise tests were performed to explore the seizure per day data without assuming a specific underlying distribution. Like the ANOVA, these methods also indicated that the Compound I 1 mg/kg and CTEP groups exhibit decreased seizure per day rates compared to the Untreated group (p<0.05 for both), whilst the RO6807794 (PAM) group exhibits increased seizure per day rates compared to the Untreated group (p<0.05).

GLM suitable for Discrete data: The GLM analysis performed was suitable for discrete (count) data and is presented in Table 9 using geometric means and 95% confidence intervals for the five treatment groups and comparisons against the Untreated group, expressed as ratios of geometric means and 95% confidence intervals. None of the groups exhibit statistically significantly lower seizure per day rates compared to the Untreated group, but the RO6807794 treatment group exhibits statistically significantly increased seizure per day rates compared to the Untreated group (p<0.001).

TABLE 6
Generalized Linear Model analysis on Discrete (count) Data for Seizure per day

| | Geometric | Lower | Upper | Ratio of | Lower | Upper | Unadjusted | % |
|---|---|---|---|---|---|---|---|---|
| | Mean | 95% CI | 95% CI | Means | 95% CI | 95% CI | P value | Change |
| Compound I | 0.910 | 0.483 | 1.712 | 1.85 | 0.71 | 4.81 | 0.2004 | 85.38 |
| 0.3 mg/kg | | | | | | | | |
| RO6807794 | 2.490 | 1.699 | 3.648 | 5.07 | 2.26 | 11.4 | 0.0002 | 407.47 |
| CTEP | 0.375 | 0.161 | 0.871 | 0.76 | 0.25 | 2.31 | 0.6285 | −23.56 |
| Compound I | 0.263 | 0.089 | 0.776 | 0.54 | 0.15 | 1.96 | 0.3406 | −46.32 |
| 1 mg/kg | | | | | | | | |
| Untreated | 0.491 | 0.240 | 1.002 | | | | | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of treating Dravet syndrome (DVS), comprising administering to a subject in need thereof a composition comprising a therapeutically effective amount of a therapeutic agent, wherein the therapeutic agent is a compound of Formula I: 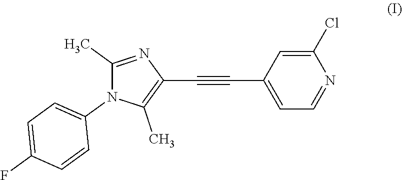 [SMILES: Cc1nc(C#Cc2ccnc(Cl)c2)c(C)n1-c1ccc(F)cc1]
or a pharmaceutically acceptable salt thereof.

2. The method of claim 1, wherein administering comprises administering the therapeutic in its free base form.

3. The method of claim 1, wherein administering comprises administering the therapeutic in the form of a pharmaceutically acceptable salt thereof.

4. The method of any one of claims 1-3, wherein the administering of the therapeutic comprises administering the therapeutic agent in an amount of about 0.05 mg to about 5 mg.

5. The method of any one of claims 1-4, wherein the administering of the therapeutic comprises administering the therapeutic in an amount of about 0.1 mg to about 4 mg.

6. The method of any one of claims 1-5, wherein the administering of the therapeutic comprises administering the therapeutic in an amount of about 0.5 mg to about 3.5 mg.

7. The method of any one of claims 1-6, wherein the administering of the therapeutic comprises administering the therapeutic in an amount of about 0.1 mg, about 0.5 mg, about 0.8 mg, about 1.0 mg, about 1.3 mg, about 1.5 mg, about 1.8 mg, about 2 mg, about 2.3 mg, about 2.5 mg, about 2.8 mg, about 3 mg, about 3.3 mg, about 3.5 mg, about 3.8 mg, or about 4.0 mg.

8. The method of any one of claims 1-7, wherein the administering of the therapeutic comprises administering the therapeutic once daily.

9. The method of any one of claims 1-8, wherein the subject is a human.

10. The method of any one of claims 1-9, wherein the subject is a patient.

11. The method of any one of claims 1-10, wherein after administration of the compound, the subject has one or more reduced seizure type.

12. The method of claim 11, wherein the seizure type is a convulsive seizure or a non-convulsive seizure.

13. The method of claim 12, wherein the convulsive seizure is reduced by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, or more.

14. The method of claim 12, wherein the non-convulsive seizure is reduced by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, or more.

15. The method of claim 11, wherein the seizure type is a focal onset seizure, a generalized onset seizure, or an unknown onset seizure.

16. The method of any one of claims 1-10, wherein after administration of the compound, the subject has reduced status epilepticus.

17. The method of claim 16, wherein the subject has reduced status epilepticus frequency by about 50% or more, about 55% or more, about 60% or more, about 65% or more, about 70% or more, about 75% or more, about 80% or more, about 85% or more, about 90% or more, about 95% or more, or completely eliminate seizures in the subject over a period of 10 days, 20 days, 30 days, 50 days, 84 days, or more.

18. The method of any one of claims 1-10, wherein after administration of the compound, the subject's hospitalization visits by about 25% or more, about 50% or more, about 75% or more, or completely eliminate hospitalization visits due to seizures.

19. The method of any one of claims 1-10, wherein after administration of the compound, a need by the subject for rescue medication is reduced by about 25% or more, about 50% or more, about 75% or more, or completely eliminate the need for rescue medication.

20. The method of any one of claims 1-10, wherein therapeutic efficacy of the treatment is determined by assessing improvement based on Clinical Global Impression-Improvement (CGI-I) Rating Score.

21. The method of any one of claims 1-10, wherein therapeutic efficacy of the treatment is determined by assessing improvement based on the Quality of Life in Childhood Epilepsy (QOLCE) Score.

22. The method of any one of claims 1-10, wherein therapeutic efficacy of the treatment is determined by assessing improvement based on the Overall Quality of Life Score From the Pediatric Quality of Life Inventory™ (PedsQL) Score.

23. The method of any one of claims 1-10, wherein therapeutic efficacy of the treatment is determined by assessing improvement based on the PedsQL Family Impact Module Score.

24. The method of any one of claims 1-10, wherein therapeutic efficacy of the treatment is determined by assessing improvement based on the EuroQOL-5 Dimensions-5 Levels scale.

25. The method of any one of claims 1-10, wherein therapeutic efficacy of the treatment is determined by assessing improvement based on the Hospital Anxiety and Depression Scale (HADS).

26. The method of any one of claims 1-10, wherein therapeutic efficacy of the treatment is determined by assessing improvement in seizure-free interval.

27. The method of claim 1, wherein the therapeutic efficacy of the treatment is determined by assessing a reduction in Seizure frequency.

28. The method of claim 1, wherein the therapeutic efficacy of the treatment is determined by assessing an increase in Seizure freedom.

29. The method of claim 1, wherein the therapeutic efficacy of the treatment is determined by assessing an increase in time to first seizure.

30. The method of claim 1, wherein the therapeutic efficacy of the treatment is determined by assessing a reduction in total seizure burden.

31. The method of claim 1, wherein the therapeutic efficacy of the treatment is determined by assessing a reduction in total seizure per day.

32. The method of any one of claims 1-31, wherein the therapeutic effect of the treatment is determined by:
a. a reduction of the overall seizure rate by 50% or higher;
b. a reduction of the convulsive seizure frequency;
c. an increase in the likelihood of being free from seizure;
d. an improvement on behavioral or cognitive symptoms; or
e. longest seizure free period.

33. A method of treating Dravet syndrome, comprising administering to a subject in need thereof a composition comprising a therapeutically effective amount of an mGlu5 negative allosteric modulator (NAM), or a pharmaceutically acceptable salt thereof, wherein the mGlu5 NAM is a compound of Formula I: 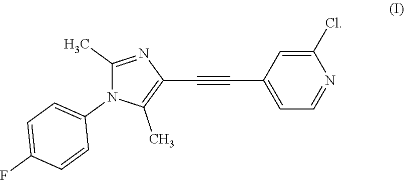 [SMILES: Cc1nc(C#Cc2ccnc(Cl)c2)c(C)n1-c1ccc(F)cc1]
